(19) United States
(12) Patent Application Publication
(10) Pub. No.: US 2009/0112041 A1
(43) Pub. Date: Apr. 30, 2009

(54) Process to Reduce the Pour Point of a Waxy Paraffinic Feedstock

(76) Inventors: Gilbert Robert Bernard Germaine, Petit-Couronne (FR); Francois Panissaud, Petit-Couronne (FR)

Correspondence Address: SHELL OIL COMPANY, P O BOX 2463, HOUSTON, TX, 772522463, US

(21) Appl. No.: 11/922,662

(22) PCT Filed: Jun. 22, 2006
(86) PCT No.: PCT/EP2006/063432

§ 371 Date: Dec. 20, 2007

(30) Foreign Application Priority Data
Jun. 23, 2005 (EP) 05291355.5

(51) Int. Cl. C07C 7/00

(52) U.S. Cl. 585/867; 585/864

(57) ABSTRACT

The invention relates to a process to reduce the pour point of a waxy paraffinic feedstock comprising a fraction boiling above 450° C. by diluting said feedstock with a solvent comprising an aliphatic ketone compound and an aromatic compound, wherein the volume ratio of ketone compound to aromatic compound is lower than 0.7:1, chilling the mixture to a temperature at which wax is caused to precipitate, physically removing the wax from an oil phase and recovering an oil product having a lower pour point than the waxy paraffinic feedstock, wherein at least part of the waxy paraffinic feedstock is derived from Fischer-Tropsch synthesis products.

The invention relates to an improved process to reduce the pour point of a waxy paraffinic feedstock by means of solvent dewaxing.

WO-A-02/46333 describes a process wherein a residual fraction of a partly hydroisomerised Fischer-Tropsch derived wax is subjected to a solvent dewaxing step in order to obtain a haze free base oil. The solvent dewaxing process is described in WO-A-02/46333 as to comprise mixing of a waxy hydrocarbon stream with a solvent, typically comprising a ketone and an aromatic, chilling the mixture to cause the wax crystals to precipitate, and separating the wax by filtration, a d recovering the solvent from the wax and the dewaxed oil filtrate. According to the description the solvent dewaxing step is preferably performed using a mixture of methyl ethyl ketone (MEK) and toluene in a weight ratio of 0.7:1 to 1:1 (corresponding to a volume ratio of 0.75:1 of MEK/toluene for a specific gravity of 0.805 at 20° C. for MEK and 0.865 for toluene at 20° C.) as preferred solvent blend for the heavier bright stock type of base oils.

Applicants found that when solvent dewaxing a waxy paraffinic feed according to the process disclosed in WO-A-02/46333 a waxy a low yield to the final base oil product is obtained.

The object of the present invention is to provide a process to prepare haze free and high viscous grade base oils in a high yield.

This object is achieved with the following process. Process to reduce the pour point of a waxy paraffinic feedstock comprising a fraction boiling above 450° C. by diluting said feedstock with a solvent comprising an aliphatic ketone compound and an aromatic compound, wherein the volume ratio of ketone compound to aromatic compound is lower than 0.7:1, chilling the mixture to a temperature at which wax is caused to precipitate, physically removing the wax from an oil phase and recovering an oil product having a lower pour point than the waxy paraffinic feedstock, wherein at least part of the waxy paraffinic feedstock is derived from Fischer-Tropsch synthesis products.

Applicants found that when the dewaxing process is performed in the above-described manner surprisingly a much higher base oil yield is obtained than disclosed for the process of WO-A-02/46333.

Conventional Solvent dewaxing processes for petroleum derived waxy feeds have been described for instance in U.S. Pat. No. 5,360,530, U.S. Pat. No. 5,494,566, U.S. Pat. No. 4,989,674 and FR-A-2124138. In particular U.S. Pat. No. 5,360,530 and U.S. Pat. No. 5,494,566 teach that the use of a solvent with high ketone content is beneficial, in particular in view of the differential between filtration temperature and pour point of the dewaxed oil. It was highly surprising in view of this teaching that when a Fischer-Tropsch derived waxy paraffinic feedstock was subjected to the solvent dewaxing treatment according to the present invention, a dewaxed oil with a pour point lower than the pour point of the waxy paraffinic feedstock could be obtained in a high yield, while maintaining filterability of the waxy mixture with a solvent of high aromatic content.

The waxy paraffinic feedstock will be comprised of wax and oil. Wax is defined as the part of the feed which will precipitate under controlled conditions. The wax content as used in the description is measured according to the following procedure. 1 weight part of the to be measured oil fraction is diluted with 4 parts of a (50/50 vol/vol) mixture of methyl ethyl ketone and toluene, which is subsequently cooled to −20° C. The mixture is subsequently filtered at −20° C. The wax is removed from the filter and any remaining solvent and oil in said wax is removed before weighing the wax. The weight fraction of this wax on the total feed is the wax content.

The waxy paraffinic feedstock will comprise a fraction which boils above 450° C., preferably above 550° C. It is this high boiling fraction, which will yield the viscous base oils. If such a heavy waxy material is subjected to the present process, an oil product may be obtained having a kinematic viscosity at 100° C. greater than 10 mm²/sec.

The presence of lower boiling compounds is permissible. The lower boiling oil component can be separated from the dewaxed oil after the pour point reducing treatment according this invention. Preferably more than 50 wt % boils above 450° C., more preferably more than 70 wt % boils above 450° C. and even more preferably more than 90 wt % boils above 450° C. in order to avoid having to separate high volumes of or any lower boiling oil components after the pour point reducing step.

The wax content of the waxy feedstock is preferably below 50 wt %, more preferably below 35 wt %. The lower limit is preferably above 5 wt %. In a most preferred embodiment the wax content is between 10 and 35 wt %. A minimal amount of wax is required in order to operate a solvent dewaxing step in an optimal manner.

The waxy paraffinic feedstock will be comprised substantially of paraffins. Applicants found that especially the yield to base oils is improved using the process according the present invention when starting from said substantially paraffinic base oils. In this boiling range it has been found difficult to quantify the paraffin content. In order to qualify a feed as paraffinic one should determine the viscosity index (VI) of the oil component of the feed. The oil should then be first isolated according to the procedure to determine wax content as described above. If the VI of the oil is greater than 120, preferably greater than 130 the feed is qualified as paraffinic.

The waxy paraffinic feedstock is preferably obtained by partly hydroisomerising a paraffin wax feed. Such a paraffin wax feed is at least in part a paraffin wax as obtained in a Fischer-Tropsch synthesis process. Preferably the waxy paraffinic feed is prepared by (a) hydroisomerisation of a Fischer-Tropsch synthesis product, and (b) isolating one or more fuel products and a distillation residue comprising the waxy paraffinic feedstock.

If the wax content of the residue is not within the above preferred ranges a preferred further reduction of the wax content is achieved by contacting the residue with a hydroisomerisation catalyst under hydroisomerisation conditions. The hydroisomerisation catalyst may be a platinum or silica-alumina catalyst as for example described in WO-A-02/070627 or preferably a zeolites based catalyst as described in for example US-A-2004/0065588, WO-A-2001/007538 or EP-A-536325.

The feedstock is preferably a distillation residue of an effluent of such a hydroisomerisation step. This residue is advantageous because it comprises the most viscous molecules as obtainable from such a hydroisomerisation process. It thus enables one to prepare the desired more viscous base oils. If such a residue would be catalytically dewaxed a less preferred hazy base oil is obtained as for example illustrated in US-A-2004/0065588. A hazy base oil is here defined as a base oil having a cloud point which is at least 25° C. higher than the pour point of the oil. By using the process according to the present invention it is possible to obtain a haze free base oil in a high yield starting from such a residual type of feed.

Partly hydroisomerised, preferably residual, feedstocks as prepared from a Fischer-Tropsch wax are well known. Examples are the feed to the deep cut distillation step of the process disclosed in WO-A-03033622, the feed to the solvent dewaxing step as disclosed in WO-A-02/46333, the residual product as obtained in the vacuum distillation step as disclosed in US-A-2004/0065588, the intermediate and partly dewaxed product as obtained by contacting a Fischer-Tropsch wax with a platinum/ZSM-48 type catalyst as disclosed in WO-A-2004/033607, the so-called heavy base oil precursor fraction as disclosed in WO-A-2004/007647 and the so-called ‘residue’ as disclosed in the examples of WO-A-02/070627.

In the process according the present invention the waxy paraffinic feedstock is diluted with a solvent. The solvent comprises an aliphatic ketone compound and an aromatic compound. Examples of suitable ketone compounds are C₃-C₆ ketones, suitably dimethyl ketone (acetone), diethyl ketone, methyl ethyl ketone, methylisobutylketone or methyl-n-propylketone. Preferably methyl ethylketone (MEK) is used. The aromatic compound is preferably an aromatic compound having a boiling point of below 170° C., more preferably C₆-C₁₀ aromatic hydrocarbons, for example benzene, ethylbenzene, o, p or m-dimethylbenzene or their mixtures and preferably toluene.

Preferably the dilution step is performed at an elevated temperature, more preferably above 0° C. and even more preferably above 20° C., most preferably above 50° C. It has been found advantageous to have a visibly clear mixture of solvent and waxy paraffinic feedstock before chilling said mixture to the dewaxing temperature. The temperature will thus be chosen such that a clear mixture is obtained, wherein the mixture becomes clearer by increasing the temperature. Accordingly, the subject invention also provides for a process, wherein the dilution step for a given solvent blend is performed at a temperature at which the mixture becomes clear, i.e. at which the waxy paraffinic feedstock is dissolved. The upper limit of the temperature will depend on the solvent mixture chosen. Practically the dilution will be performed at a temperature below the boiling point of the solvent used. Preferably the temperature at which the dilution is performed is between 50 and 80° C., more preferably between 55 and 75° C.

The volume ratio of ketone compound to aromatic compound is lower than 0.7:1, preferably lower than 0.65:1. A volume ratio of ketone compound to aromatic compound of 0.7:1 may conveniently also be expressed as 1:1.429. It has been found that when more volume of aromatic compound is applied a higher oil yield is achieved. The preferred volume ratio is greater than 1:1.429, and more preferably greater than 1:1.5. More preferably, the volume ratio is greater than 1:1.9, yet more preferably more than 1:2, again more preferably more than 1:2.5. There is also a preferred upper limit for this ratio. A higher volume of aromatic compound may result in hazy base oils and/or a less efficient filtration. The volume ratio is therefore preferably below 1:19, more preferably below 1:10, more preferably below than 1:6 and yet more preferably below 1:5.

The overall solvent to waxy feed volume ratio (also generally referred to as the solvent oil ratio) will depend largely on the wax content of the feed, the viscosity of the feed, and the desired pour point of the dewaxed oil product. Usually, the overall solvent to waxy feed volume ratio is in the range of from 10:1 to 5:1, typically between 6:1 and 3:1.

The diluted waxy paraffinic feed is chilled to a temperature at which the wax compounds will precipitate. The chilling temperature will determine the resulting pour and cloud point of the oil. The chilling or temperature reduction is preferably performed at a low rate in order to obtain a wax precipitate, which can be easily filtered. More preferably this rate is below 5° C. per minute, more preferably below 3° C. per minute and preferably above 0.5° C. per minute. Applicants have surprisingly found that the pour point of the resulting base oil is lower than the chilling temperature applied. This is especially observed when starting from the above referred to residual feedstocks. Without being bound to the following theory it is believed that a small amount of very heavy compounds determine the pour point of the waxy paraffinic feedstock. These compounds can be present when starting from relatively heavy Fischer-Tropsch waxes as illustrated in, for example, the process described in WO-A-02/070627. These compounds will, most likely, be more easily removed in the process according to the invention resulting in an oil which can have a lower pour point than the ‘chilling temperature’ applied in the dewaxing step itself. For most applications of the base oil as obtained by the present process will suitably have a pour point of below 0° C. and preferably below −5° C. The lower limit will be −50° C. The chilling temperature is preferably below 0° C., more preferably below −10° C. and even more preferably below −20° C.

The precipitated wax compounds are physically removed from the oil, preferably by filtering through a filter cloth which can be made of textile fibres, such as cotton; porous metal cloth; or cloth made of synthetic materials. The above described solvent dewaxing may be performed in apparatuses known for solvent dewaxing lubricating base oils as described in Lubricant Base Oil and Wax Processing, Avilino Sequeira, Jr, Marcel Dekker Inc., New York, 1994, Chapter 7. Any solvent remaining in the wax compounds or the oils can be conveniently removed by evaporation. In practice, this is done by evaporation under vacuum, for example by heating the oil to 150° C., and of reduced pressure. Accordingly, recovery of the oil product preferably includes removal of any solvent left in the oil product after removal of the precipitated wax.

In the process according the present invention also a wax is obtained. It has been found that such a wax is a relatively soft wax, which may be used for various purposes. The soft wax as obtained with the above process has preferably a congealing point as determined by ASTM D 938 of between 85 and 120 and more preferably between 95 and 120° C. and a PEN at 43° C. as determined by IP 376 of more than 0.8 mm and preferably more than 1 mm. The wax is further characterized in that it preferably comprises less than 1 wt % aromatic compounds and less than 10 wt % naphthenic compounds, more preferably less than 5 wt % naphthenic compounds.

If low oil contents in the wax by-product are desired it may be advantageous to perform an additional de-oiling step. De-oiling processes are well known and are for example described in Lubricant Base Oil and Wax Processing, Avilino Sequeira, Jr, Marcel Dekker Inc., New York, 1994, pages 162-165. After de-oiling the wax preferably has a oil content of between 0.1 and 2 wt %. The lower limit is not critical. Values of above 0.5 wt % may be expected, but lower values can be achieved depending on the method in which the wax is obtained. Most likely the oil content will be between 1 and 2 wt %. The kinematic viscosity at 150° C. of the wax is preferably higher than 8 cSt and more preferably higher than 12 and lower than 18 cSt.

The haze free base oil will preferably have a kinematic viscosity at 100° C. of above 10 cSt, preferably above 14 cSt which viscosity may range up to 30 cSt and even above. The viscosity index is suitably above 120 and preferably above 130 and more preferably above 140. A haze free base oil is determined by its cloud point. A haze free base oil according to this invention has a cloud point as determined by ASTM D 2500 of near the pour point and below 0° C., preferably below −10° C. and more preferably below −15° C. The difference in cloud point and pour point is preferably below 25° C. and more preferably below 15° C.

### EXAMPLE 1

From a hydroisomerised Fischer-Tropsch wax an atmospheric distillation residue was isolated having the properties as listed in Table 1. The atmospheric residue was further separated under high vacuum to obtain a vacuum residue having the properties as listed in Table 1.

TABLE 1

| | | Atmospheric | Vacuum |
|---|---|---|---|
| FEED | | residue | residue |
| d70/4 | | 0,7874 | n.d. |
| Pour Pt | ° C. | >+48 | n.d. |
| Congealing point | ° C. | +56 | +85 |
| (ASTM D-938) | | | |
| N | mg/kg | <1 | <1 |
| S | mg/kg | <2 | <2 |
| Vk @ 100° C. | mm²/s | n.d. | 22,57 |
| Wt % recovered at | | | |
| 400° C. | Wt % | 29.7 | 0 |
| 450° C. | Wt % | 43.2 | 0.8 |
| 500° C. | Wt % | 53.8 | 9.8 |
| 550° C. | Wt % | 66.5 | 32.5 |
| 600° C. | Wt % | 78.6 | 52 |
| 650° C. | Wt % | 87.8 | 68.8 |
| 700° C. | Wt % | 94.3 | 81.9 |
| 740° C. | Wt % | 96.5 | 89.7 |
| WAX CONTENT* | Wt % | 34 | 41 |

*Dewaxing temperature @ −20° C.;
n.d. = not determined

The above vacuum residue was contacted with a hydroisomerisation catalyst consisting of 0.7 wt % platinum, 25 wt % ZSM-12 and a silica binder in order to further reduce the wax content of the vacuum residue. The reaction conditions were 40 bar hydrogen, reactor temperature of 338° C., weight hourly space velocity=1 kg/l·h, and a hydrogen gas rate of 500 Nl/kg feed.

The effluent of the hydroisomerisation reaction as described above was diluted at 70° C. with a methyl ethylketone/toluene solvent mixture having the volume ratios as listed in Table 3. All solutions were clear before cooling. The amount of solvent employed was about 3 to 4 times the amount of waxy feed. The temperature was reduced to −20° C. at a rate of 25° C./hour. Filtration was performed at −20° C. Solvent was removed from the oil product obtained under vacuum to less than 100 ppm. The results are listed in Table 3.

Examples 1a and 1d are comparative examples.

TABLE 3

| Example | | 1-a | 1-b | 1-c | 1-d |
|---|---|---|---|---|---|
| MEK:toluene | (vol/vol) | 1:19 | 1:6 | 1:3 | 1:1 |
| volume ratio | | | | | |
| Maximum | Wt % | 96 | 93 | 92 | 65 |
| theoretical | | | | | |
| oil yield * | | | | | |
| Filtration | | slow | slow; | Good; | Acceptable; |
| rate | | | filter | dry | oi1y cake |
| | | | plugged | cake | |
| OIL Properties | | n.a. | | | |
| density d20/4 | | | 0.8344 | 0.8344 | 0.8338 |
| Pour Pt | ° C. | ** | −27° C. | −24 | −27 |
| Vk40 | mm²/sec | ** | 134.7 | 133.4 | 120 |
| Vk100 | mm²/sec | ** | 18.14 | 17.96 | 16.51 |
| VI | | ** | 150 | 150 | 149 |
| Aspect | | Hazy | Clear | clear | clear |

* This is the maximum oil yield, which could be achieved. However in practical commercial operation this can only be achieved when also the rate of filtration is good and the filter does not plug. For that reason the results at example 1-c are the most favourable in this experiment, since they reflect the actually achieved oil yield.
** Due to the fact that the oil obtained was hazy no further properties were measured.

1. A process to reduce the pour point of a waxy paraffinic feedstock comprising a fraction boiling above 450° C., said process comprising
diluting said feedstock with a solvent comprising an aliphatic ketone compound and an aromatic compound;
wherein the volume ratio of ketone compound to aromatic compound is lower than 0.7:1,
chilling the mixture to a temperature at which wax is caused to precipitate; and
physically removing the wax from an oil phase and recovering an oil product having a lower pour point than the waxy paraffinic feedstock;
wherein at least part of the waxy paraffinic feedstock is derived from Fischer-Tropsch synthesis products.

2. The process according to claim 1, wherein the volume ratio of ketone compound to aromatic compound is between 1:1.5 and 1:10.

3. The process according to claim 1, wherein the aliphatic ketone compound is selected from the group consisting of dimethyl ketone, diethyl ketone, methyl ethyl ketone, methylisobutylketone, and methyl-n-propylketone.

4. The process according to claim 1, wherein the ketone is methyl ethyl ketone.

5. The process according to claim 1, wherein the aromatic compound is toluene.

6. The process according to claim 1, wherein the waxy paraffinic feedstock has a wax content of between 10 and 50 wt %.

7. The process according to claim 6, wherein the wax content of the feed is below 35 wt %.

8. The process according to claim 1, wherein the dilution step for a given solvent blend is performed at a temperature at which the mixture becomes clear prior to chilling.

9. The process according to claim 1, wherein the mixture is chilled to a temperature between −50 and −10° C.

10. The process according to claim 1, wherein the waxy paraffinic feed has a temperature of between 50 and 80° C. on diluting the feedstock with the solvent.

11. The process according to claim 1, wherein the waxy paraffinic feed comprises more than 80 wt % of compounds boiling above 450° C.

12. The process according to claim 1, wherein the waxy paraffinic feed is prepared by
(a) hydroisomerisation of a Fischer-Tropsch synthesis product; and
(b) isolating one or more fuel products and a distillation residue comprising the waxy paraffinic feedstock.

13. The process according to claim 12, wherein the wax content of the residue is reduced to a value between 10 and 50 wt % by contacting the feed with a hydroisomerisation catalyst under hydroisomerisation conditions.
